(19) United States
(12) Patent Application Publication
(10) Pub. No.: US 2020/0215000 A1
(43) Pub. Date: Jul. 9, 2020

(54) COMBINATION THERAPY OF PHOSPHATE BINDERS AND VITAMIN K

(71) Applicant: KAYDENCE PHARMA AS, Oslo (NO)

(72) Inventors: Daniel H. ROSENBAUM, Conshohocken, PA (US); Lee M. TECHNER, Ardmore, PA (US)

(21) Appl. No.: 16/733,985

(22) Filed: Jan. 3, 2020

Related U.S. Application Data
(60) Provisional application No. 62/788,523, filed on Jan. 4, 2019.

(51) Int. Cl. A61K 31/122; A61K 45/06

(52) U.S. Cl. CPC A61K 31/122; A61K 45/06

(57) ABSTRACT

A method of treating chronic kidney disease including administering to a subject in need thereof a combination of a vitamin K and a phosphate binder.

### CROSS-REFERENCE TO RELATED APPLICATION

This application claims priority to U.S. Provisional Patent Application No. 62/788,523, filed Jan. 4, 2019, the disclosure of which is incorporated herein by reference in its entirety

### TECHNICAL FIELD

The present disclosure is directed to the use of a combination therapy comprising a phosphate binder and vitamin K. The combination may be used, for example, for treating patients suffering from chronic kidney disease; for reducing high serum phosphate levels, also known as hyperphosphatemia; for protecting vasculature from calcification; for reversing vascular calcification; for reducing non-bone calcium levels; for mitigating bone degeneration; for reducing the dose of phosphate binders to achieve the same benefits, or reduce side effects thereof; and/or for reducing cardiovascular incidents (MACE), morbidity or mortality.

### BACKGROUND OF THE DISCLOSURE

Patients suffering from chronic kidney disease (CKD) are prone to a number of various complications. For example, CKD patients often suffer from high serum phosphate levels, also known as hyperphosphatemia. CKD is also linked to calcium deposits in the soft tissue, particularly the soft tissue of the cardiovascular system, which often results in cardiovascular disease. CKD patients also suffer from weakened bones due to calcium being pulled out of the bones as well as excessive calcium levels in the blood and soft tissue. These effects are often observed in later stage 4 and 5 (dialysis) patients, although they are also observed in stage 3 and renal transplant patients.

Many CKD patients are prescribed phosphate binders in conjunction with dietary phosphate restrictions in order to reduce the risk of and/or treat hyperphosphatemia. Phosphate binders have been shown to effectively reduce the absorption of phosphate, thereby reducing serum phosphate levels. However, the use of phosphate binders may itself result in unintentional and harmful consequences. In particular, phosphate binders may also bind with certain fat soluble molecules, including vitamin K. This effect is problematic, as the dietary phosphate restrictions often recommended for subjects suffering from hyperphosphatemia generally restrict consumption of the very limited potential sources of vitamin K, a vitamin which has been linked to bone strength and arterial health. In particular, vitamin K is required for carboxylation of two bone matrix proteins necessary for normal bone metabolism. It is also required to prevent hemorrhaging by activating blood-clotting factors. As such, by binding with vitamin K, phosphate binders may further deplete a likely already low (i.e., subclinically deficient) level of vitamin K in these subjects.

### BRIEF DESCRIPTION OF THE DISCLOSURE

The present disclosure is directed to the use of a combination therapy comprising a phosphate binder and vitamin K. The combination may be used, for example, for treating patients suffering from CKD; for reducing high serum phosphate levels, also known as hyperphosphatemia; for protecting vasculature from calcification; for reversing vascular calcification; for reducing non-bone calcium levels; for mitigating bone degeneration; for reducing the dose of phosphate binders to achieve the same benefits, or reduce side effects thereof; and/or for reducing cardiovascular incidents (MACE), morbidity or mortality.

### DETAILED DESCRIPTION OF THE DISCLOSURE

The present disclosure is directed to a method of administering a combination of a phosphate binder and vitamin K to a subject, wherein the combination functions to reduce serum phosphate levels in the subject; to protect vasculature from calcification; to reverse vascular calcification; to reduce non-bone calcium levels; to mitigate bone degeneration; to reduce the dose of phosphate binders to achieve the same benefits, or reduce side effects thereof; and/or to reduce cardiovascular incidents (MACE), morbidity or mortality. The subject may be a patient suffering from CKD. In certain embodiments, the subject may be a patient suffering from CKD and cardiovascular disease (CVD). In certain embodiments, the patient may be undergoing concomitant dialysis treatment. The method may be used to treat CVD in a patient suffering from CKD, or prevent the onset of CVD in a CKD patient.

The combination according to the present disclosure comprises vitamin K. Those skilled in the art will understand that vitamin K and derivatives thereof refer to one or more compounds of Formula 1 and their pharmaceutically or nutritionally acceptable salts:

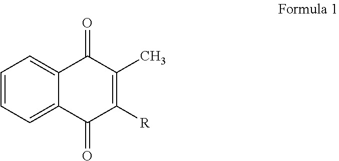

wherein R may be any covalently linked organic group including polyisoprenoid residues, esters, ethers, and thiol adducts. According to some aspects, the vitamin K comprised by the combination may be a vitamin K₂, i.e., a menaquinone, selected from the group consisting of short-chain menaquinones (i.e., MK-1, MK-2, MK-3, and MK-4), long-chain menaquinones (i.e., MK-5, MK-6, MK-7, MK-8, and MK-9), and combinations thereof. Those skilled in the art will understand that menaquinones are abbreviated MK-n, wherein M represents menaquinone, K represents vitamin K, and n represents the number of isoprenoid side chain residues. According to some aspects, the vitamin K may comprise or consist of MK-7.

The combination according to the present disclosure also comprises a phosphate binder. As used herein, the term “phosphate binder” refers to a compound capable of binding to phosphate in the human body, thereby reducing phosphate absorption by the body. Examples of phosphate binders according to the present disclosure include, but are not limited to, aluminum hydroxide, aluminum salts, bixalomer, calcium acetate, calcium carbonate, ferric citrate, lanthanum carbonate, magnesium carbonate, nicotinic acid, olestilan, sevelamer carbonate, sevelamer hydrochloride, sucroferric oxyhydroxide, and combinations thereof.

The method comprises administering a first amount of vitamin K in combination with a second amount of phosphate binder. It should be understood that a “combination” or “in combination” as used herein may refer to simultaneous and/or sequential administration. For example, the method may comprise administering the first amount of vitamin K followed by administering the second amount of phosphate binder before or during the time that the first amount of vitamin K is (or becomes) active in the body, or vice versa. According to some aspects, the method may comprise administering the first amount of vitamin K simultaneously or about simultaneously with the second amount of phosphate binder.

The first and second amounts may be administered in one or more daily doses. Those skilled in the art will understand that a “dose” refers to the quantity of an agent administered at a particular point in time. According to some aspects, the first amount of vitamin K may be delivered in a single daily dose or may be delivered over the course of multiple doses per day. For example, the first amount of vitamin K may be administered in one, two, three, four, five, or more daily doses, wherein each dose contains the same or a different amount of vitamin K with respect to one or more other doses. Similarly, the second amount of phosphate binder may be administered in one, two, three, four, five, or more daily doses, wherein each dose contains the same or a different amount of phosphate binder with respect to one or more other doses. According to some aspects, each dose of vitamin K may independently be administered simultaneously with or sequentially with respect to a dose of phosphate binder. According to some aspects, each dose of vitamin K may be administered simultaneously with or about simultaneously with a dose of phosphate binder.

The first amount of vitamin K may be a therapeutically effective amount. According to some aspects, the therapeutically effective amount of vitamin K may refer to an amount of vitamin K that, when administered, improves endothelial dysfunction, improves arterial flexibility, reduces arterial stiffness and/or reverses calcification of a blood vessel in a mammal; reduces, reverses, and/or eliminates bone weakening and/or degeneration; reduces, reverses, and/or eliminates risk of cardiovascular incidents (MACE), morbidity, and/or mortality; and/or reduces, reverses, and/or eliminates the negative impacts of phosphate binder administration (such as side effects).

According to some aspects, the therapeutically effective amount of vitamin K may depend on one or more factors, such as the age of the subject and/or the degree of CKD, hyperphosphatemia, vascular calcification, CVD, blood calcium levels, soft tissue calcium levels, and/or a combination thereof and/or the therapeutically effective amount of phosphate binder (as discussed below).

According to some aspects, the therapeutically effective amount of vitamin K may be an amount sufficient to reduce and/or eliminate one or more negative effects of administering the second amount of phosphate binder. For example, the phosphate binder may interact with certain fat soluble molecules, including, for example, vitamin K. Administering the phosphate binder alone to a subject in need thereof may therefore reduce the subject's vitamin K to an unacceptable level, which may, for example, increase the subject's risk of vascular calcification, cardiovascular disease stemming at least in part from vascular calcification, and/or a combination thereof. According to some aspects, the therapeutically effective amount of vitamin K may be an amount sufficient to provide an acceptable level of vitamin K to the subject, such as reducing or avoiding one or more side effects with phosphate binders seen in the absence of the vitamin K administration. According to some aspects, the acceptable level of vitamin K may correspond to a level wherein the risk of vascular calcification, cardiovascular disease stemming at least in part from vascular calcification, risk of cardiovascular incidents (MACE), morbidity, and/or mortality, and/or a combination thereof is reduced and/or eliminated when compared with the subject's risk without vitamin K administration. According to some aspects, the acceptable level of vitamin K may correspond to a level wherein a subject's vascular calcification, cardiovascular disease stemming at least in part from vascular calcification, symptoms thereof, and/or a combination thereof is reduced and/or eliminated. According to some aspects, the acceptable level of vitamin K may correspond to a level wherein bone weakening and/or degeneration, and/or unacceptable non-bone levels of calcium (e.g., in the blood and/or in soft tissue) is reduced and/or eliminated.

According to some aspects, the acceptable level of vitamin K may be determined by determining an acceptable level of one or more other biomarkers in a subject. For example, the acceptable level of vitamin K may be a vitamin K level that provides an acceptable level of inactive MGP, i.e., desphospho-uncarboxylated MGP (dp-ucMGP). In general, lower levels of dp-ucMGP are more acceptable than higher levels, as higher levels are associated with vascular calcification, particularly in subjects with CKD. Specifically, without wishing to be bound by a particular theory, it is believed that the activated form of matrix γ-carboxyglutamic acid protein (MGP) at least in part reduces vascular calcification and non-bone calcium levels, and vitamin K is at least partially responsible for carboxylation of the protein to its active form. Thus, dp-ucMGP (i.e., the inactive form) levels may correspond with vitamin K levels, and in particular, unacceptably high levels of dp-ucMGP may correspond with an unacceptable level of vitamin K.

According to some aspects, an acceptable level of dp-ucMGP (i.e., a vitamin K sufficient level) may be no more than about 700 pmol/L, optionally no more than about 650 pmol/L, optionally no more than about 600 pmol/L, optionally no more than about 550 pmol/L, optionally no more than about 500 pmol/L, optionally no more than about 450 pmol/L, optionally no more than about 400 pmol/L, and optionally no more than about 350 pmol/L. According to some aspects, an acceptable level of dp-ucMGP may be between about 300 and 700 pmol/L, optionally between about 250 and 650 pmol/L, optionally between about 300 and 600 pmol/L, optionally between about 350 and 550 pmol/L, and optionally between about 400 and 500 pmol/L.

According to some aspects, the therapeutically effective amount of vitamin K may be an amount that increases a subject's kidney filtration and/or excretion when compared with the subject's kidney filtration and/or excretion without vitamin K administration. In this way, the vitamin K may reduce, reverse, and/or eliminate unacceptable serum phosphate levels (i.e., hyperphosphatemia), independently or synergistically with the phosphate binder. The therapeutically effective amount of vitamin K may be an amount that reduces a subject's bone degeneration when compared with the subject's bone degeneration without vitamin K administration.

According to some aspects, the therapeutically effective amount of vitamin K may be an amount of vitamin K that lowers the therapeutically effective amount of phosphate binder. In particular, the therapeutically effective amount of vitamin K may be an amount wherein a lesser amount of the phosphate binder is required to reduce and/or eliminate hyperphosphatemia in a subject when compared with the amount of the phosphate binder required to reduce and/or eliminate hyperphosphatemia in the subject when vitamin K is not administered. According to some aspects, the therapeutically effective amount of vitamin K may be an amount wherein a lesser amount of the phosphate binder is required to reduce and/or eliminate vascular calcification, cardiovascular disease stemming at least in part from vascular calcification, bone weakening and/or degeneration, unacceptable non-bone levels of calcium (e.g., in the blood and/or in soft tissue), and risk of cardiovascular incidents (MACE), morbidity, and/or mortality, and/or a combination thereof in a subject when compared with the amount of the phosphate binder required to provide a comparable effect when vitamin K is not administered.

According to some aspects, the dosage of vitamin K may be between about 1 and 2000 mcg/day, optionally between about 100 and 1500 mcg/day, optionally between about 200 and 1400 mcg/day, optionally between about 300 and 1200 mcg/day, and optionally between about 360 and 1200 mcg/day. According to some aspects, the dosage of vitamin K may be at least 360 mcg/day. According to some aspects, the dosage of vitamin K may be no more than about 2000 mcg/day, optionally no more than about 1200 mcg/day. According to some aspects, the dosage of vitamin K may be between 300 and 2000 mcg/day.

The second amount of phosphate binder may be a therapeutically effective amount. As used herein, the term “therapeutically effective amount” refers to an amount of an agent that, when administered to a subject, results in an improvement, reversal, or remediation of a disease and/or symptoms thereof. According to some aspects, the therapeutically effective amount of phosphate binder may refer to an amount of phosphate binder that, when administered, reduces, reverses, and/or eliminates unacceptable serum phosphate levels (i.e., hyperphosphatemia). As used herein, an unacceptable serum phosphate level, or hyperphosphatemia, refers to a serum phosphate concentration of greater than about 4.5 mg/dL (or greater than about 1.46 mmol/L). The therapeutically effective amount of phosphate binder may be an amount required to treat hyperphosphatemia in a subject when administered in combination with vitamin K, that is, to reduce the subject's serum phosphate concentration to 4.5 mg/dL or less (i.e., 1.46 mmol/L or less). According to some aspects, the therapeutically acceptable amount of phosphate binder is less than the amount of phosphate binder required to provide one or more of the above effects in a subject when the phosphate binder is administered alone and/or not in combination with vitamin K.

According to some aspects, the dosage of phosphate binder may be between 1 and 8000 mg/day, optionally between about 500 and 5000 mg/day, optionally between about 800 and 4500 mg/day. According to some aspects, each dose of the phosphate binder may be taken with a meal, for example, between about 500 and 200 mg/meal, optionally between about 800 and 1500 mg/meal.

The therapeutically effective amount of phosphate binder may depend on one or more factors, such as the age of the subject and/or the degree of CKD, hyperphosphatemia, vascular calcification, CVD, bone weakening, blood calcium levels, soft tissue calcium levels, and/or a combination thereof.

It is believed that the combination therapy of the invention provides synergistic results, that is, the effects of the combination are greater than (or provide benefits that are different than) either therapy alone. In some embodiments, this synergistic effect could be determined, for example, by measuring carotid-femoral pulse wave velocity (cfPWV), estimated glomerular filtration rate (eGFR) and/or dp-ucMGP levels with either therapy alone as compared to the combination.

According to some aspects, the combination may be administered enterally, parenterally, topically, or a combination thereof. It should be understood that enteral administration includes oral, buccal, enteral, and intragastric administration. Parenteral administration includes any form of administration in which the combination is absorbed into the blood stream without involving absorption via the intestines. Examples of parenteral administration include, but are not limited to intramuscular, intravenous, intraperitoneal, intraocular, subcutaneous, and intraarticular administration, and combinations thereof.

The method comprises administering the combination to a subject in need thereof. According to some aspects, the subject may suffer from hyperphosphatemia. According to some aspects, the subject may suffer from compromised kidney function and/or chronic kidney disease (CKD). The subject may suffer from calcium deposits in soft tissue, particularly the cardiovascular system; cardiovascular disease; weakened bones, particularly weakened bones from calcium being pulled therefrom; and/or excessive calcium levels in the blood and/or soft tissue, which may or may not be an effect of hyperphosphatemia. In certain preferred embodiments, the subject may be a patient suffering from CKD and CVD. In certain embodiments, the patient may be undergoing concomitant dialysis treatment. The method may be used to treat CVD in a patient suffering from CKD, or prevent the onset of CVD in a CKD patient. The subject may be a mammal such as a human, pet animal (e.g., dogs, cats), laboratory animal (e.g., rats, mice), or a farm animal (e.g., sheep, horses, cows).

The present disclosure is also directed to a composition comprising the combination as described herein. The composition may be in the form of a tablet (coated or uncoated), capsule (hard or soft), dragee, lozenge, oral solution, suspension, dispersion, syrup, sterile parenteral preparation, and/or a combination thereof. It should be understood that the composition may comprise one or more forms as described herein, wherein each form includes one or both components (i.e., the vitamin K and the phosphate binder) of the combination.

The composition may additionally include pharmaceutically acceptable additives, carriers, excipients, or a combination thereof. Examples of excipients include, but are not limited to, diluents such as calcium carbonate, sodium carbonate, lactose, calcium phosphate, and sodium phosphate; granulating and disintegrating agents such as cornstarch or alginic acid; binding agents such as starch gelatin or acacia; effervescents; lubricating agents such as magnesium stearate, stearic acid, and talc; and combinations thereof. Examples of additives including, but are not limited to, preservatives, chelating agents, effervescing agents, natural and artificial sweeteners, flavoring agents, coloring agents, taste masking agents, acidulants, emulsifiers, thickening agents, suspending agents, dispersing agents, wetting agents, antioxidants, and combinations thereof.

The composition may be provided as a fortified food or beverage. Examples of fortified food and beverages include, but are not limited to, juice drinks, dairy drinks, powdered drinks, sports drinks, mineral water, soy beverages, hot chocolate, malt drinks, biscuits, bread, crackers, confectioneries, chocolate, chewing-gum, margarines, spreads, yogurts, breakfast cereals, snack bars, meal replacements, protein powders, desserts, medical nutrition tube feeds, nutritional supplements, and combinations thereof.

The present disclosure is also directed to a kit containing the compositions as described herein along with instructions for administering the combination as described herein.

This written description uses examples to disclose the invention, including the preferred embodiments, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. Aspects from the various embodiments described, as well as other known equivalents for each such aspect, can be mixed and matched by one of ordinary skill in the art to construct additional embodiments and techniques in accordance with principles of this application.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference. Moreover, nothing disclosed herein is intended to be dedicated to the public.

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C.

As used herein, the term “about” and “approximately” are defined to being close to as understood by one of ordinary skill in the art. In one non-limiting embodiment, the term “about” and “approximately” are defined to be within 10%, preferably within 5%, more preferably within 1%, and most preferably within 0.5%.

1. A method of treating chronic kidney disease comprising administering to a subject in need thereof a combination of a vitamin K in a first amount and a phosphate binder in a second amount.

2. The method according to claim 1, wherein the vitamin K comprises a vitamin K₂.

3. The method according to claim 2, wherein the vitamin K₂ comprises MK-7.

4. The method according to claim 1, wherein the first amount and the second amount are sufficient to reduce the subject's serum phosphate level to 4.5 mg/dL or less.

5. The method according to claim 1, wherein the first amount and the second amount are sufficient to reduce the subject's dp-ucMGP level to no more than about 700 pmol/L.

6. The method according to claim 1, wherein the first amount and the second amount are sufficient to prevent onset of cardiovascular disease in the subject.

7. The method according to claim 1, wherein the first amount and the second amount are sufficient to prevent vascular calcification in the subject and/or reduce the subject's vascular calcification.

8. The method according to claim 1, wherein the first amount and the second amount are sufficient to reduce the subject's non-bone calcium level.

9. The method according to claim 1, wherein the first amount and the second amount are sufficient to reduce a rate of bone degeneration in the subject.

10. The method according to claim 1, wherein the vitamin K and the phosphate binder are administered simultaneously.

11. A method of treating cardiovascular disease comprising administering to a subject in need thereof a combination of a vitamin K in a first amount and a phosphate binder in a second amount, wherein the cardiovascular diseases is characterized at least by vascular calcification.

12. A synergistic combination of a vitamin K in a first amount and a phosphate binder in a second amount.

13. The combination according to claim 8, wherein the vitamin K comprises a vitamin K₂.

14. The combination according to claim 8, wherein the vitamin K₂ comprises MK-7.
